(19) United States
(12) Patent Application Publication
(10) Pub. No.: US 2018/0153735 A1
(43) Pub. Date: Jun. 7, 2018

(54) COLD AND WARM FOMENTATION DEVICE USING THERMOELECTRIC ELEMENT

(71) Applicant: CAMEL CO, LTD., Chungcheongbuk-do (KR)

(72) Inventor: Je-Kyung SHIM, Chungcheongbuk-do (KR)

(21) Appl. No.: 15/822,399

(22) Filed: Nov. 27, 2017

(30) Foreign Application Priority Data
Dec. 7, 2016 (KR) 10-2016-0166084

(51) Int. Cl. A61F 7/00; A61F 7/02

(52) U.S. Cl. CPC A61F 7/007; A61F 7/02; A61F 2007/0088; A61F 2007/0078; A61F 2007/0096; A61F 2007/0075; A61F 2007/0228

(57) ABSTRACT

There is provided a cold and warm fomentation device including a cylindrical case, an upper portion and a lower portion of which are opened, a far-infrared ray ceramic plate formed of a material emitting a far-infrared ray and coupled to the case, a thermoelectric element such as a Peltier element, configured to apply heat to the far-infrared ray ceramic plate, a cover coupled to an upper portion of the case to fix the far-infrared ray ceramic plate, and a temperature adjusting circuit unit configured to adjust a direction and an intensity of a current supplied to the thermoelectric element.

### CROSS-REFERENCE TO RELATED APPLICATIONS

This application claims the priority of Korean Patent Application No. 10-2016-0166084 filed on Dec. 7, 2016, in the Korean Intellectual Property Office, the disclosure of which is incorporated herein by reference.

### FIELD

The present invention relates to a cold and warm fomentation device using a thermoelectric element, and more particularly, to a cold and warm fomentation device using a thermoelectric element, which may irradiate, to an affected part of a user, a far-infrared ray emitted from a far-infrared ceramic plate together with warm air or cold air having a temperature of 6 to 42° C. formed by a thermoelectric element such as a Peltier element.

### BACKGROUND

In general, a fomentation device heats or freezes water as a cold/warm medium, which is accommodated in a space inside the device, to come into contact with an affected part so as to perform fomentation. In the fomentation device according to the related art, a large amount of time is consumed for the heating or the freezing, and it is not easy for a user to maintain a desired temperature for a desired time.

Accordingly, in order to overcome the disadvantages of the above-described fomentation device, studies have been conducted in the past to employ a thermoelectric element assembly that performs a heat generation function and a heat absorption function in the cold and warm fomentation device.

As an example, “a cold and warm-combined fomentation device using a thermoelectric element” is disclosed in Korean Patent Laid-Open Publication No. 10-2004-0021829. The cold and warm fomentation device includes a warm fomentation unit in contact with one end of the thermoelectric element, a cold fomentation unit in contact with the other end of the thermoelectric element, and temperature sensors provided in the fomentation units to measure the temperatures of the fomentation units, respectively, in which the temperatures of the fomentation units may be adjusted according to temperatures set by a user by using values of the temperature sensors.

However, the temperature sensors measure the temperatures of the fomentation units themselves in contact with the thermoelectric element, which are different from the temperature of a skin of the user. That is, because the temperatures of the fomentation units themselves are set and are adjusted, and the temperatures are not equal to the temperature of the affected part directly felt by the user, user's satisfaction deteriorates, and when the cold and warm fomentation device is used for a long time, there is a concern that skin damages such as low-temperature burns and erythema are caused without user's recognition.

Thus, it is eager to develop a cold and warm fomentation device in which a user may set the temperature of an affected part, and the temperature of the affected part may be adjusted, and which may, for example, perform cold fomentation at a skin temperature of 35° C. or warm fomentation at a skin temperature of 39° C.

In particular, in the cold and warm-combined fomentation device according to the related art, because water is used as a cold/warm medium and metal is used as a heat transfer medium, electromagnetic waves may be generated in the device, which may adversely affect user's health.

[Patent document]: (Patent document 0001) Korean Patent Laid-Open Publication No. 10-2004-0021829 (Publication date: 2004 Mar. 11.)

### SUMMARY

The present invention has been conceived to solve the above-described problems of the related art, and an aspect of the present invention provides a cold and warm fomentation device using a thermoelectric element, which may irradiate, to an affected part of a user, a far-infrared ray together with warm air or cold air having a temperature of 6 to 42° C.

According to an aspect of the present invention, there is provided a cold and warm fomentation device including a cylindrical case, an upper portion and a lower portion of which are opened and which has a support step transversely extending inward from a lower end of the case, a container-type far-infrared ray ceramic plate formed of a material emitting a far-infrared ray, having a stopper step transversely extending outward from an upper end of the far-infrared ray ceramic plate, having a top-opened accommodation space, and coupled to the case, a thermoelectric element fixed to an upper portion of the far-infrared ray ceramic plate to apply heat to the far-infrared ray ceramic plate, a cover coupled to an upper portion of the case, and a temperature adjusting circuit unit configured to adjust a direction and an intensity of a current supplied to the thermoelectric element.

In the cold and warm fomentation device according to an embodiment of the present invention, a bottom surface of the far-infrared ray ceramic plate may be a curved surface convexly protruding as it goes toward a center of the bottom surface.

In the cold and warm fomentation device according to the embodiment of the present invention, the far-infrared ray ceramic plate may have a compounding ratio of 33 to 37 wt % of kaolin, 27 to 33 wt % of spodumene, 7 to 10 wt % of talc, 7 to 10 wt % of clay, 7 to 10 wt % of feldspar, and 11 to 15 wt % of sericite.

The cold and warm fomentation device according to the embodiment of the present invention may further include one or more belt hooks formed on an upper surface of the cover, and fixing belts fitted with the belt hooks to fix the cold and warm fomentation device to a human body.

In the cold and warm fomentation device according to the embodiment of the present invention, a blower fan installed in the thermoelectric element may be further provided, and one or more blowing ports may be formed in the far-infrared ray ceramic plate or the cover.

In the cold and warm fomentation device according to the embodiment of the present invention, a fixing panel may be arranged on the far-infrared ray ceramic plate, an outer edge of the fixing panel may be fixed between the far-infrared ray ceramic plate and the cover, and a power supply unit, a controller, and a temperature adjusting unit may be fixed to the fixing panel.

In the cold and warm fomentation device according to the embodiment of the present invention, the temperature adjusting circuit unit may include a power supply unit configured to supply electric power, a power switch configured to switch “on/off” electric power supplied from the power supply unit, a temperature sensor arranged between the far-infrared ceramic plate and the thermoelectric element, a temperature adjusting switch configured to set a specific temperature, a cold fomentation mode, or a warm fomentation mode, a controller configured to control a direction and an intensity of a current supplied to the thermoelectric element according to the specific temperature, the cold fomentation mode, or the warm fomentation mode set by the temperature adjusting switch, and a temperature adjusting unit configured to adjust the direction and the intensity of the current supplied from the power supply unit to the thermoelectric element under a control of the controller.

In the cold and warm fomentation device according to the embodiment of the present invention, the temperature adjusting circuit unit may further include a timer configured to provide time information to the controller, and an operation time setting unit configured to set an operation time, in which the controller counts the time information provided by the timer to perform a control to supply the current from the power supply unit to the thermoelectric element for the operation time set by the operation time setting unit.

In the cold and warm fomentation device according to the embodiment of the present invention, the controller may adjust the direction and the intensity of the current supplied to the thermoelectric element such that the cold fomentation mode, the warm fomentation mode, or a concurrent cold and warm fomentation mode are selectively performed.

In the cold and warm fomentation device according to the embodiment of the present invention, when the power supply unit is a battery, a charging terminal installed in the case or the cover such that a portion of the charging terminal is exposed to the outside, and a charge adjusting unit provided between the charging terminal and the power supply unit to adjust charging of the electric power supplied to the power supply unit through the charging terminal may be provided.

According to an embodiment of the present invention, a far-infrared ray ceramic plate configured to emit a far-infrared ray when heat is applied to a portion in direct contact with a human body, that is, an affected part, is configured, and a thermoelectric element configured to selectively supply warm air or cold air is attached to the far-infrared ray ceramic plate, so that a user may perform cold fomentation or warm fomentation while irradiating a far-infrared ray to an affected part.

Further, according to the present invention, the bottom surface of the far-infrared ray ceramic plate is a curved surface convexly protruding as it goes to the center thereof, so that adhesiveness with the affected part may be improved. Further, the far-infrared ray ceramic plate is formed of kaolin, spodumene, talc, clay, feldspar, and sericite, so that the far-infrared ray ceramic plate emits a far-infrared ray, and is easily formed, and the strength of the far-infrared ray ceramic plate may be maintained. Further, belt hooks in which fixing belts may be fitted are formed in the cover, so that the cold and warm fomentation device may be fixed to an affected part of a human body such as a back or a leg. Furthermore, a blower fan is installed in the case to be adjacent to the thermoelectric element to circulate inner air, so that the thermoelectric element may be prevented from being overheated.

Further, according to the present invention, a fixing panel is inserted into the case, and a power supply unit, a control unit, a temperature adjusting unit, and the like are arranged, so that the cold and warm fomentation device may be used anywhere and anytime even without separate connection between the cold and warm fomentation device and an external power source, fomentation at a temperature desired by the user, a cold fomentation mode, and a warm fomentation mode may be selectively performed, and a far-infrared ray emitting time, a cold fomentation time, and a warm fomentation time may be adjusted such that fomentation may be performed for a time desired by the user.

Further, according to the present invention, the power supply unit configured to supply electric power to the thermoelectric element, the blower fan, and the like is a battery, and the battery is recharged often, so that the cold and warm fomentation device may be used anywhere and anytime regardless of locations.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view illustrating a cold and warm fomentation device using a thermoelectric element according to an embodiment of the present invention;

FIG. 2 is a front view illustrating the cold and warm fomentation device of FIG. 1 including a fixing belt;

FIG. 3 is a side sectional view illustrating the cold and warm fomentation device of FIG. 1;

FIG. 4 is a block diagram illustrating a temperature adjusting circuit unit of the cold and warm fomentation device of FIG. 1; and

FIG. 5 is a flowchart for explaining an operation state of the cold and warm fomentation device of FIG. 1.

### DETAILED DESCRIPTION

Hereinafter, a cold and warm fomentation device using a thermoelectric element according to various embodiments of the present invention will be described in detail with reference to the accompanying drawings.

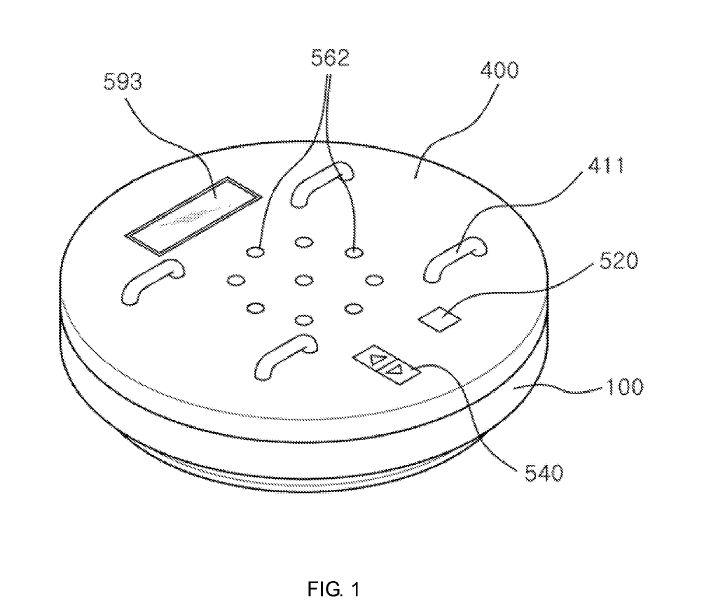

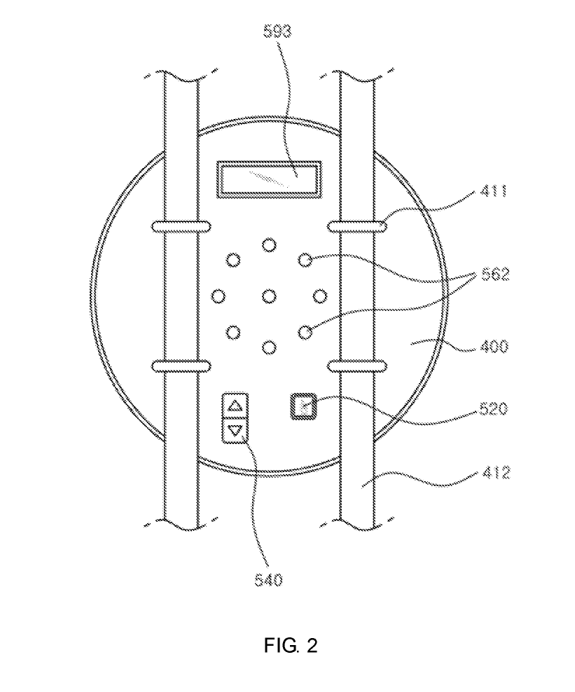

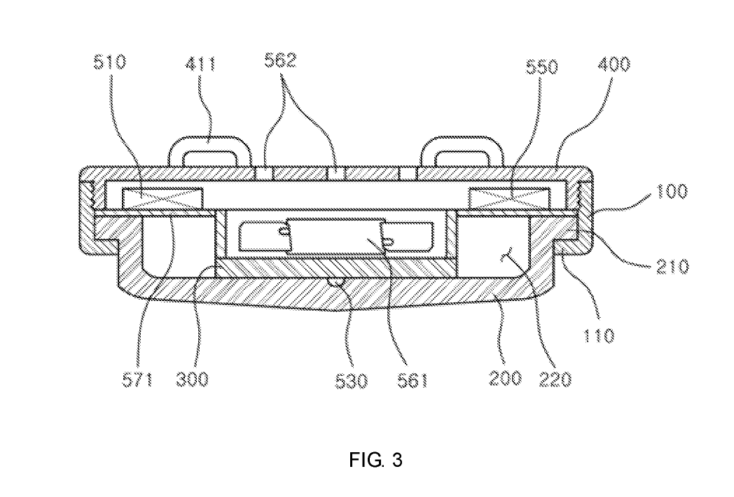

FIG. 1 is a perspective view illustrating a cold and warm fomentation device using a thermoelectric element according to an embodiment of the present invention, FIG. 2 is a front view illustrating the cold and warm fomentation device of FIG. 1 including a fixing belt, and FIG. 3 is a side sectional view illustrating the cold and warm fomentation device of FIG. 1.

The cold and warm fomentation device using a thermoelectric element according to an embodiment of the present invention includes a cylindrical case 100, a container-shaped far-infrared ray ceramic plate 200, a thermoelectric element 300 such as a Peltier element, a cover 400, and a temperature adjusting circuit unit 500, as illustrated in FIGS. 1 to 3.

The cylindrical case 100, which is formed of plastic or the like, has an inner space, an upper portion and a lower portion of which are opened to accommodate configurations of the far-infrared ray ceramic plate 200, the thermoplastic element 300, a blower fan 561, a fixing panel 571, and the temperature adjusting circuit unit 500. Further, a support step 110 transversely extends inward from a lower end of the cylindrical case 100 to support the far-infrared ray ceramic plate 200 coupled to the corresponding case 100 so as to prevent the far-infrared ray ceramic plate 200 from escaping to the outside.

The far-infrared ray ceramic plate 200 is formed of a material emitting a far-infrared ray, and is formed in a container shape. The above far-infrared ray ceramic plate 200 has a stopper step 210 which corresponds to the support step 110 of the case 100 and transversely extends outward from an upper end of the far-infrared ray ceramic plate 200, has a top-opened accommodation space 220 configured to accommodate the thermoelectric element 300 and the blower fan 561, and is coupled to the case 100. Thus, when the far-infrared ray ceramic plate 200 is coupled to the case 100, the stopper step 210 of the far-infrared ray ceramic plate 200 is stopped by the support step 110 of the case 100, so that the far-infrared ray ceramic plate 200 is not separated from the case 100.

In the above embodiment, as the bottom surface of the far-infrared ray ceramic plate 200 is a curved surface convexly protruding as it goes toward the center thereof, when the cold and warm fomentation device according to the embodiment is fixed to an affected part of the user, the bottom surface of the far-infrared ray ceramic plate 200 comes into close contact with the affected part, so that loss of a far-infrared ray, warm air, and cold air transferred to the affected part may be minimized.

In the above embodiment, the far-infrared ray ceramic plate 200 may be formed of kaolin, spodumene, talc, clay, feldspar, and sericite. It is preferable that a compounding ratio thereof is a combination of 33 to 37 wt % of kaolin, 27 to 33 wt % of spodumene, 7 to 10 wt % of talc, 7 to 10 wt % of clay, 7 to 10 wt % of feldspar, and 11 to 15 wt % of sericite.

That is, it can be identified that when the compounding ratio for forming the far-infrared ray ceramic plate 200 is a combination of 33 wt % of kaolin, 12 wt % of spodumene, 10 wt % of talc, 7 wt % of clay, 8 wt % of feldspar, and 30 wt % of sericite, a combination of 33 wt % of kaolin, 17 wt % of spodumene, 10 wt % of talc, 7 wt % of clay, 8 wt % of feldspar, and 25 wt % of sericite, or a combination of 33 wt % of kaolin, 22 wt % of spodumene, 10 wt % of talc, 7 wt % of clay, 8 wt % of feldspar, and 20 wt % of sericite, thermal resistance of the far-infrared ray ceramic plate 200 deteriorates.

Further, it can be identified that when the compounding ratio for forming the far-infrared ray ceramic plate 200 is a combination of 33 wt % of kaolin, 27 wt % of spodumene, 10 wt % of talc, 7 wt % of clay, 8 wt % of feldspar, and 15 wt % of sericite, all performance of the far-infrared ray ceramic plate 200 is proper.

However, it can be identified that when the compounding ratio for forming the far-infrared ray ceramic plate 200 is a combination of 33 wt % of kaolin, 32 wt % of spodumene, 10 wt % of talc, 7 wt % of clay, 8 wt % of feldspar, and 10 wt % of sericite, thermal resistance of the far-infrared ray ceramic plate 200 increases, when the compounding ratio for forming the far-infrared ray ceramic plate 200 is a combination of 33 wt % of kaolin, 37 wt % of spodumene, 10 wt % of talc, 7 wt % of clay, 8 wt % of feldspar, and 5 wt % of sericite, far-infrared ray performance of the far-infrared ray ceramic plate 200 deteriorates, and when the compounding ratio for forming the far-infrared ray ceramic plate 200 is a combination of 33 wt % of kaolin, 27 wt % of spodumene, 10 wt % of talc, 10 wt % of clay, 5 wt % of feldspar, and 15 wt % of sericite, a sintering problem occurs, that is, a firing temperature increases.

Further, when the compounding ratio for forming the far-infrared ray ceramic plate 200 is a combination of 28 wt % of kaolin, 27 wt % of spodumene, 10 wt % of talc, 10 wt % of clay, 10 wt % of feldspar, and 15 wt % of sericite, a sintering problem occurs, that is, a firing temperature decreases.

Thus, in order to maximize performance of the far-infrared ray ceramic plate 200, it is preferable that the compounding ratio for forming the far-infrared ray ceramic plate 200 is a combination of 33 wt % of kaolin, 27 wt % of spodumene, 10 wt % of talc, 7 wt % of clay, 8 wt % of feldspar, and 15 wt % of sericite.

The thermoelectric element 300, which supplies warm air or cold air desired by the user to the far-infrared ray ceramic plate 200, that is, emits heat to the far-infrared ray ceramic plate 200 or absorbs heat of the far-infrared ray ceramic plate 200 by the Peltier effect, is inserted into the accommodation space 220 of the far-infrared ray ceramic plate 200, and is fixed to an upper portion of the far-infrared ray ceramic plate 200.

Here, the “Peltier effect” refers to a phenomenon which was discovered by J. C. A. Peltier in France in 1834 and in which when a direct current (DC) voltage is applied to opposite ends of different two elements, heat is absorbed on one surface thereof and heat is generated on the other surface thereof depending on a direction of a current. In this way, an element using the Peltier effect is referred to as a “Peltier element”. The basic principle of the Peltier effect is that electrons require energy to move between two metals having potential difference, the energy required herein is obtained by taking energy of the metals. When the direction in which the current flows changes, flow of the electrons and holes changes, and the surfaces emitting/absorbing heat are also reversed.

A thermoelectric element such as the above-described Peltier element has advantages in that because the element has a solid structure, reliability is high, the element may be used semi-permanently, the same thermoelectric element provides two functions including heating and cooling, the affected part may be cooled to a temperature that is lower than the ambient temperature, and when an appropriate control system is provided, the temperature may be controlled in a unit that is more precise than 0.05° C.

The cover 400, which is formed of plastic or the like, is coupled to an upper portion of the case 100 to protect configurations of the far-infrared ray ceramic plate 200, the thermoelectric element 300, the blower fan 561, the fixing panel 571, and the temperature adjusting circuit unit 500, which are coupled to or accommodated in the inner space of the case 100 and the accommodation space 220 of the far-infrared ray ceramic plate 200.

The above case 100 and the above cover 400 are screw-coupled to each other or are coupled to each other through a fastening unit such as a bolt and a nut.

The temperature adjusting circuit unit 500 may adjust the direction and the intensity of the current supplied to the thermoelectric element 300 to allow the thermoelectric element 300 to generate heat required by the user, that is, to allow the thermoelectric element 300 to perform a warm fomentation mode or a cold fomentation mode through a heat emitting effect or a heat absorbing effect.

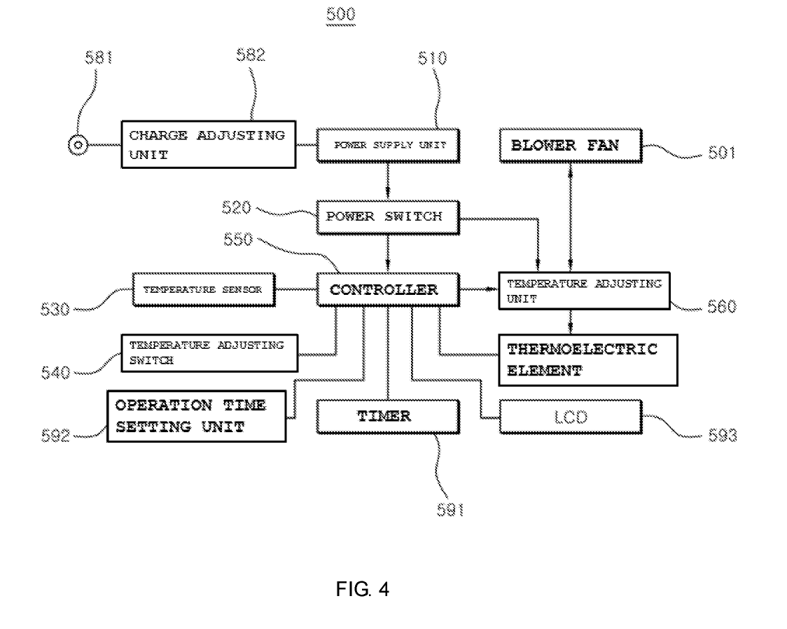

As illustrated in FIG. 4, the above-described temperature adjusting circuit unit 500 includes a power supply unit 510 configured to supply electric power, a power switch 520 configured to switch “on/off” electric power supplied from the power supply unit 510, a temperature sensor 530 arranged between the far-infrared ray ceramic plate 200 and the thermoelectric element 300 to detect the temperature of warm air or cold air generated by the thermoelectric element 300 and transferred to an affected part of a human body through the far-infrared ray ceramic plate 200, a temperature adjusting switch 540 configured to set a specific temperature desired by the user or set the cold fomentation mode or the warm fomentation mode having a predetermined temperature band, a controller 550 configured to control the direction and the intensity of the current supplied to the thermoelectric element 300 depending on the specific temperature, the cold fomentation mode or the warm fomentation mode set by the temperature adjusting switch 540, and a temperature adjusting unit 560 configured to adjust the direction and the intensity of the current supplied from the power supply unit 510 to the thermoelectric element 300 under a control of the controller 550, so that the user may foment the affected part by the warm air having the temperature desired by the user or in the predetermined cold fomentation mode or the predetermined warm fomentation mode through the heat emitting effect and the heat absorbing effect of the thermoelectric element 300 while irradiating a far-infrared ray.

According to another embodiment of the present invention, four belt hooks 411 bent in a “U” shape are fixed to the upper surface of the cover 400 in two rows through adhesive such as glue or a fastening unit such as a screw, fixing belts 412 having fastening units such as Velcro tape, provided at ends thereof, are fitted in the belt hook 411 arranged in two rows, so that the user may fix the cold and warm fomentation device to a back, an arm, a leg, or the like where the affected part is located.

Further, according to another embodiment of the present invention, the blower fan 561 is installed in the thermoelectric element 300, and a plurality of blowing ports 562 configured to suction external air and discharge warm air or cold air generated by the thermoelectric element 300 to the outside, by the blower fan 561, is formed in the far-infrared ray ceramic plate 200 or the cover 400, so that the thermoelectric element 300 may be prevented from being overheated.

Further, according to another embodiment of the present invention, a fixing panel 571 is arranged on an upper portion of the far-infrared ray ceramic plate 200, and an outer edge of the fixing panel 571 is fitted and fixed between the far-infrared ray ceramic plate 200 and the cover 400, so that the user may fix the power supply unit 100, the controller 550, and the temperature adjusting unit 560 to the fixing panel 571, and accommodate the power supply unit 100, the controller 550, and the temperature adjusting unit 560 in the case 100.

Further, according to the embodiment of the present invention, the temperature adjusting circuit unit 500 includes a timer 591 configured to provide, to the controller 550, time information on the standard time for control of the cold and warm fomentation device, and an operation time setting unit 592 configured to set an operation time desired by the user. The controller 550 performs a control to count the time information provided by the timer 590, and supply a current from the power supply unit 510 to the thermoelectric element 300 for the operation time set by the operation time setting unit 592, so that the user may set the cold and warm fomentation device to operate in a desired temperature, in the warm fomentation mode, or in the cold fomentation mode for a desired time.

That is, the user may set the operation time by manipulating the operation time setting unit 592, and may adjust the direction and the intensity of the current supplied to the thermoelectric element 300 by manipulating the temperature adjusting switch 540 such that the cold and warm fomentation device may selectively perform fomentation at the temperature desired by the user, the cold fomentation mode, the warm fomentation mode, or a concurrent cold and warm fomentation mode by the controller 550.

According to another embodiment, when the power supply unit 510 is a battery, a charging terminal 581 connected to the power supply unit 510, that is, the battery, is installed in the case 100 or the cover 400 such that a portion of the charging terminal 581 is exposed to the outside, and a charge adjusting unit 582 configured to adjust charging of electric power supplied to the power supply unit 510 through the charging terminal 582 is provided between the charging terminal 581 and the power supply unit 510, so that the cold and warm fomentation device may be operated by using commercial electric power and may be operated by using electric power of the battery anywhere and anytime regardless of locations and times.

In the drawing, not-described reference numeral 593 is a liquid crystal display (LCD) configured to show various operation states of the cold and warm fomentation device.

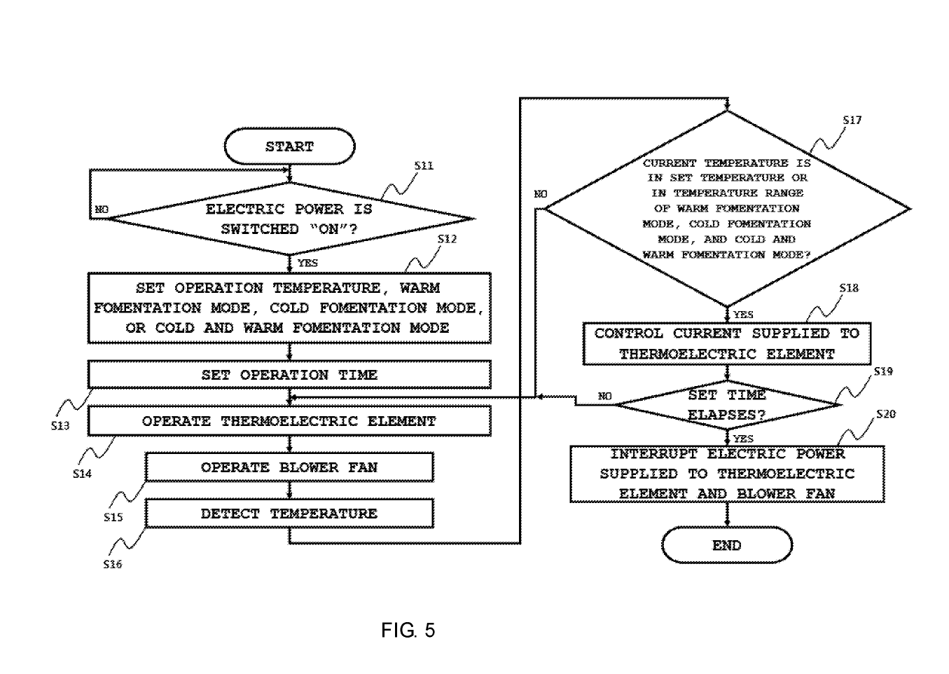

FIG. 5 is a flowchart for explaining an operation state of the cold and warm fomentation device of FIG. 1.

Referring to FIG. 5, when the power switch 520 is switched “on” (S11), the controller 550 sets an operation mode to a mode in which fomentation is performed at an operation temperature, the warm fomentation mode, the cold fomentation mode, or the cold and warm fomentation mode according to the temperature adjusting switch 540 (S12), and sets an operation time for which fomentation is performed at the operation temperature, or the warm fomentation mode, the cold fomentation mode, or the cold and warm fomentation mode according to manipulation of the operation time setting unit 592 (S13).

When the operation mode and the operation time are set as above, the controller 550 operates the temperature adjusting unit 560 to supply the electric power supplied from the power supply unit 510 to the thermoelectric element 300, and at the same time, to the blower fan 561, so as to operate the thermoelectric element 300 and the blower fan 561 (S14, S15).

In this way, when the thermoelectric element 300 and the blower fan 561 are operated, the controller 550 compares a temperature detected by the temperature sensor 530 (S16) with a predetermined temperature or a temperature range in which the warm fomentation mode, the cold fomentation mode, or the cold and warm fomentation mode may be performed (S17), to adjust the direction and the intensity of the current supplied to the thermoelectric element 300 (S18).

Further, the controller 550 counts a time for which the mode in which fomentation is performed at the temperature set by the user, the warm fomentation mode, the cold fomentation mode, or the cold and warm fomentation mode is performed by operating the thermoelectric element 300 and the blower fan 561, to identify whether a predetermined time elapses (S19), repeatedly controls the operations of the thermoelectric element 300 and the blower fan 561 as above when it is identified that the predetermined time doesn't elapse, but terminates the operation of the cold and warm fomentation device by interrupting the electric power supplied to the thermoelectric element 300 and the blower fan 561 when it is identified that the set time elapses (S20).

Although a state in which cold and warm fomentation is performed while the far-infrared ray is irradiated to the affected part has been described as an example in the above embodiment, the present invention is not limited thereto, and cold/hot mist by an herbal extract suitable for the hot and cold fomentation may be sprayed depending on the symptom on the affected part, so that the fomenting effect may be doubled.

Here, the cold mist composition, that is, a herbal medicine included in the herbal extract, consists of 0.3 g of menthol, 0.2 g of Angelica gigas, 0.2 g of Rehmanniae Radix Preparata, 0.1 g of Paeonia japonica, 0.1 g of Chinese Liquorice, and 0.1 g of pine needles, and 100 wt % of the herbal medicine consists of 30 wt % of menthol, 20 wt % of Angelica gigas, 20 wt % of Rehmanniae Radix Preparata, 10 wt % of Paeonia japonica, 10 wt % of Chinese Liquorice, and 10 wt % of pine needles as the contents of the ingredients.

Further, the warm mist composition, that is, a herbal medicine included in the herbal extract, consists of 10 g of gardenia seeds, 10 g of Turmeric, 10 g of ginger, 10 g of Korean angelica root, 10 g of Cnidium officinale, 5 g of Chinese Liquorice, 15 g of peppermint, 2 g of camphor, 8 g of Salvia miltiorrhiza, and 20 g of mugwort, and 100 wt % of the herbal medicine consists of 10 wt % of gardenia seeds, 10 wt % of Turmeric, 10 wt % of ginger, 10 wt % of Korean angelica root, 10 wt % of Cnidium officinale, 5 wt % of Chinese Liquorice, 15 wt % of peppermint, 2 wt % of camphor, 8 wt % of Salvia miltiorrhiza, and 20 wt % of mugwort as the contents of the ingredients.

The above description of the present invention is intended to be illustrative, and It can be understood by those skilled in the art to which the present invention pertains that the present invention may be modified in other specific forms without departing from the technical spirit or the essential feature of the present invention.

Therefore, it should be understood that the above-described embodiments are not restrictive but illustrative in all aspects.

For example, components described in a singular form may be implemented in a distributed form, and likewise, components described in a distributed form may be implemented in a combined form.

The scope of the present invention is defined by the appended claims, and it should be interpreted that all changes and modifications derived from the meaning and the scope of the appended claims, and equivalents thereof are included in the scope of the present invention.

What is claimed is:

1. A cold and warm fomentation device comprising:
a cylindrical case, an upper portion and a lower portion of which are opened and which has a support step transversely extending inward from a lower end of the case;
a container-type far-infrared ray ceramic plate formed of a material emitting a far-infrared ray, having a stopper step transversely extending outward from an upper end of the far-infrared ray ceramic plate, having a top-opened accommodation space, and coupled to the case;
a thermoelectric element fixed to an upper portion of the far-infrared ray ceramic plate to apply heat to the far-infrared ray ceramic plate;
a cover coupled to an upper portion of the case to fix the far-infrared ray ceramic plate; and
a temperature adjusting circuit unit configured to adjust a direction and an intensity of a current supplied to the thermoelectric element.

2. The cold and warm fomentation device of claim 1, wherein a bottom surface of the far-infrared ray ceramic plate is a curved surface convexly protruding as it goes toward a center of the bottom surface.

3. The cold and warm fomentation device of claim 1, wherein the far-infrared ray ceramic plate has a compounding ratio of 33 to 37 wt % of kaolin, 27 to 33 wt % of spodumene, 7 to 10 wt % of talc, 7 to 10 wt % of clay, 7 to 10 wt % of feldspar, and 11 to 15 wt % of sericite.

4. The cold and warm fomentation device of claim 1, further comprising:
one or more belt hooks formed on an upper surface of the cover; and
fixing belts fitted with the belt hooks to fix the cold and warm fomentation device to a human body.

5. The cold and warm fomentation device of claim 1, wherein a blower fan installed in the thermoelectric element is further provided, and one or more blowing ports are formed in the far-infrared ray ceramic plate or the cover.

6. The cold and warm fomentation device of claim 1, wherein a fixing panel is arranged on an upper portion of the far-infrared ray ceramic plate, an outer edge of the fixing panel is fixed between the far-infrared ray ceramic plate and the cover, and a power supply unit, a controller, and a temperature adjusting unit are fixed to the fixing panel.

7. The cold and warm fomentation device of claim 1, wherein the temperature adjusting circuit unit comprises:
a power supply unit configured to supply electric power;
a power switch configured to switch “on/off” electric power supplied from the power supply unit;
a temperature sensor arranged between the far-infrared ceramic plate and the thermoelectric element;
a temperature adjusting switch configured to set a specific temperature, a cold fomentation mode, or a warm fomentation mode;
a controller configured to control a direction or an intensity of a current supplied to the thermoelectric element according to the specific temperature, the cold fomentation mode, or the warm fomentation mode set by the temperature adjusting switch; and
a temperature adjusting unit configured to adjust the direction and the intensity of the current supplied from the power supply unit to the thermoelectric element under a control of the controller.

8. The cold and warm fomentation device of claim 1, wherein the temperature adjusting circuit unit further comprises:
a timer configured to provide time information to the controller; and
an operation time setting unit configured to set an operation time, and
wherein the controller counts the time information provided by the timer to perform a control to supply the current from the power supply unit to the thermoelectric element for the operation time set by the operation time setting unit.

9. The cold and warm fomentation device of claim 1, wherein the controller adjusts the direction and the intensity of the current supplied to the thermoelectric element such that the cold fomentation mode, the warm fomentation mode, or a concurrent cold and warm fomentation mode are selectively performed.

10. The cold and warm fomentation device of claim 1, wherein when the power supply unit is a battery, a charging terminal installed in the case or the cover such that a portion of the charging terminal is exposed to the outside; and a charge adjusting unit provided between the charging terminal and the power supply unit to adjust charging of the electric power supplied to the power supply unit through the charging terminal are provided.

11. The cold and warm fomentation device of claim 2, wherein the far-infrared ray ceramic plate has a compounding ratio of 33 to 37 wt % of kaolin, 27 to 33 wt % of spodumene, 7 to 10 wt % of talc, 7 to 10 wt % of clay, 7 to 10 wt % of feldspar, and 11 to 15 wt % of sericite.

12. The cold and warm fomentation device of claim 7, wherein the temperature adjusting circuit unit further comprises:
a timer configured to provide time information to the controller; and
an operation time setting unit configured to set an operation time, and
wherein the controller counts the time information provided by the timer to perform a control to supply the current from the power supply unit to the thermoelectric element for the operation time set by the operation time setting unit.

13. The cold and warm fomentation device of claim 7, wherein the controller adjusts the direction and the intensity of the current supplied to the thermoelectric element such that the cold fomentation mode, the warm fomentation mode, or a concurrent cold and warm fomentation mode are selectively performed.

14. The cold and warm fomentation device of claim 7, wherein when the power supply unit is a battery, a charging terminal installed in the case or the cover such that a portion of the charging terminal is exposed to the outside; and a charge adjusting unit provided between the charging terminal and the power supply unit to adjust charging of the electric power supplied to the power supply unit through the charging terminal are provided.
